(19) United States
(12) Patent Application Publication
(10) Pub. No.: US 2018/0055717 A1
(43) Pub. Date: Mar. 1, 2018

(54) Method and Device for Improving Visual Performance

(71) Applicant: CLEARLYVENTURE LIMITED, Berlin (DE)

(72) Inventors: Robert Lange, Dresden (DE); Uwe Kaempf, Liebstadt (DE); Sascha Seewald, Dresden (DE)

(21) Appl. No.: 15/803,825

(22) Filed: Nov. 5, 2017

Related U.S. Application Data
(63) Continuation of application No. 14/360,386, filed on Jan. 21, 2015, now abandoned.

(30) Foreign Application Priority Data
Nov. 23, 2011 (DE) 10 2011 119 361.1

(51) Int. Cl. A61H 5/00; A61B 5/00; A61B 3/113

(52) U.S. Cl. CPC A61H 5/00; A61B 5/4863; A61H 2201/5058; A61H 2201/5043; A61H 2201/5007; A61H 2201/5092; A61B 3/113

(57) ABSTRACT

An apparatus for improving a visual function, the apparatus having a first display element for displaying at least one moving grid consisting of grid elements; at least one portable closure element for the occlusion of at least one eye; a portable eye tracking device for measuring at least one characteristic value of a non-occluded eye; a first control unit having a first communication interface to the eye tracking device and a second communication interface to the at least one closure element; and a second control unit connected to the display element, with a third communication interface to the first control unit. The second control unit is adapted to provide a display to a non-occluded eye of the moving grids on the first display element controlled on the basis of the characteristic values measured by the eye tracking device.

### CROSS-REFERENCE TO RELATED APPLICATIONS

The present application is a continuation of U.S. patent application Ser. No. 14/360,386 filed by the present inventors on Jan. 21, 2015, which is a U.S. national stage of PCT/EP2012/733399 filed on Nov. 23, 2012, and which claims the benefit of the filing of German Patent Application No. 10 2011 119 361.1.

The aforementioned non-provisional patent application, PCT application, and German patent application are hereby incorporated by reference in their entirety.

### BACKGROUND OF THE INVENTION

### Field of the Invention

The present invention relates to an apparatus and a system for improving a visual function, the use of the same and a method and a computer program product for improving a visual function.

### Brief Description of the Related Art

The optokinetic nystagmus (OKN) is an involuntarily occurring movement pattern of the eyes that can be triggered, for example, by optical grids moved at a defined speed and having a defined spatial frequency and a sinusoidal brightness distribution. This movement pattern is characterized by a rapid and a slow phase. During the slow phase, the eyes follow the moving grid, and during the rapid phase, after a certain distance, jump back rapidly in order to subsequently follow the grid again. An optokinetic nystagmus can occur both in the horizontal direction, as a horizontal optokinetic nystagmus (HOKN), and in the vertical direction, as a vertical optokinetic nystagmus (VOKN), depending on whether the movement pattern of the stimulating device runs horizontally or vertically.

When the stimulation triggering this movement pattern takes place under the condition that one of the eyes is sufficiently covered, i.e. occluded, the monocular optokinetic nystagmus (MOKN) occurs in the non-occluded eye.

The OKN represents a characteristic product of certain functions of the visual system of a person. Its examination can therefore offer relevant information about possibly present limitations of the visual function, such as occurring for example in amblyopia patients. Further, the targeted stimulation of the OKN can lead to an improvement of the individual visual functions, whose interactions produce the OKN.

The term amblyopia or lazy eye designates the weakness of sight of one eye (or more rarely both eyes) which is based on an insufficient development of the visual system in early childhood. The result is a reduction of the visual acuity, which cannot or at least not be sufficiently explained by organic defects, and which continues to exist even despite optimal optical correction by means of glasses or contact lenses.

From the state of the art systems are already known for measuring and improving the contrast sensitivity and/or the visual acuity of persons in whom this contrast sensitivity and the visual acuity do not correspond to standard values (U.S. Pat. No. 8,002,409). In particular, for amblyopic persons, a classification of the amblyopia can be carried out corresponding to the hypothesis of internal and external noise. A classification according to this document is based, among other things, on the work of Andrew B. Watson “Detection and recognition of simple spatial forms” in “Physical and biological processing of images”, published by O. J. Braddick and A. C. Sleigh (Springer Verlag, Berlin, 1983). For the measuring and/or training described therein, the user must make inputs via a user interface. To produce the corresponding optical stimuli, hardware is made available which is suitable to generate on the basis of a standardized 3×8 bit color video signal a gray-scale signal with a higher resolution than 8 bits.

Other telemedical systems for measuring and improving the contrast sensitivity and/or the visual acuity of persons are known from the patents of Neurovision. Among other documents, the U.S. Pat. No. 7,004,912 and U.S. Pat. No. 7,427,138, as well as the PCT application WO 2007/043047 disclose such systems.

From the KR 1020010103223 A, a system is known that can cause an OKN reaction through a program for optokinetic nystagmi. An electro-oculographic procedure is employed for measuring and recording the eye movement. Therein an electrical voltage is measured, however which can be carried out only by correspondingly trained persons.

Further, from the document GB 1 372 988 an apparatus is known that is suitable for testing a visual acuity utilizing an optokinetic nystagmus. A cathode-ray tube is employed, the ray of which is so diverted that a vertical grid is represented. The speed, the contrast and the dimensions of the grid are varied. Besides the apparatus for producing stimuli triggering an optokinetic nystagmus, the detection of the occurrence of the optokinetic nystagmus by observation or by an electro-encephalogram signal is also disclosed. However, both variants require the additional presence of a correspondingly trained person.

Both systems known from the state of the art require the operation of the systems by a correspondingly trained person. In particular, not only the process of the measuring and/or of the training depend on the operation, but also the success of the measure can depend decisively on inputs to be made and on deficits of the same, i.e. incorrect inputs.

It is the object of the present invention to improve the visual function of a person without being dependent on operation by a correspondingly trained person.

### SUMMARY OF THE INVENTION

The present disclosure discloses an apparatus for improving a visual function, having a first display element for displaying at least one moving grid consisting of grid elements; at least one portable closure element for the respective occlusion of an eye; a portable eye tracking device, for measuring at least one characteristic value of a non-occluded eye; a first control unit having a first communication interface to the eye tracking device and a second communication interface to the at least one closure element; and a second control unit connected to the display element, having a third communication interface to the first control unit; wherein the second control unit is adapted to provide a display to a non-occluded eye of the moving grids on the first display element controlled on the basis of the characteristic values measured by the eye tracking device, and wherein the second control unit is adapted to so control the display to a non-occluded eye on the first display element that additionally attention-capturing stimuli are displayed.

The present disclosure discloses a system for improving a visual function, having at least one apparatus, and a control device with a third control unit, with a second display element, at least one input device and at least one fourth communication interface to the second control units of the at least one apparatus; wherein at least one of the second control units is further adapted to collect operating data during the operation of the corresponding apparatus and to transfer them to the third control unit of the control device, receive control information from the third control unit of the control device and control the operation of the corresponding apparatus taking account of the control information received from the third control unit of the control device, and wherein the third control unit is adapted to display on the second display element the operating data transferred from the at least one apparatus, and is adapted to send control information based on an input entered via the at least one input device to the second control unit of the corresponding apparatus.

The present disclosure discloses a method for improving a visual function, comprising the occlusion of an eye by a closure element, measuring at least one characteristic value of a non-occluded eye by an eye tracking device, and displaying to a non-occluded eye moving grids and attention-capturing stimuli on a display element, which is controlled by a control unit on the basis of the characteristic values measured by the eye tracking device.

The present disclosure discloses a computer program product for improving a visual function, wherein, when the computer program product is executed by a processor, the method according to the above method is carried out.

By the present invention, measurements can be triggered in an automated fashion, and also automated data analyses can be carried out. The resulting measurement data can also be made usable directly, thereby increasing the efficiency of improvement of the visual function. By the automatic measurement, further data can be measured that make longer-term changes of the state of the visual function reproducible.

### BRIEF DESCRIPTION OF THE DRAWINGS

There are shown:

FIG. 1 an apparatus for improving the visual function,

FIG. 2 a system for improving the visual function, and

FIG. 3 a flow chart of a method for improving the visual function.

### DETAILED DESCRIPTION

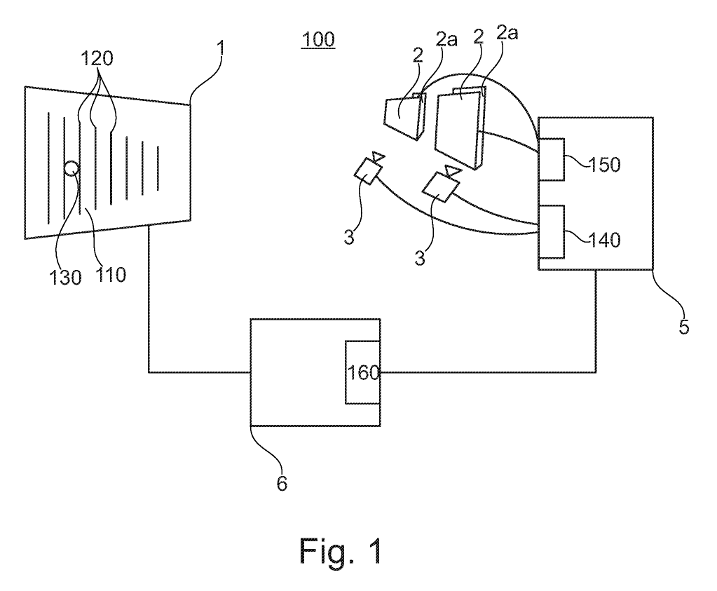

FIG. 1 shows an apparatus 100 for improving the visual function of a viewer. The apparatus 100 comprises a first display element 1. On this display element 1 there are displayed grids 110 consisting of grid elements 120, and so-called attention-capturing stimuli 130, i.e. optical-visual stimuli that capture the attention of a viewer. These attention-capturing stimuli can be of different types, can have any geometric shape and can be represented either in color or in monochrome. However, also other stimuli that are suitable to capture the attention of the viewer can be used. To be able to subject the eyes of a viewer independently of each other to the moving grids and the attention-capturing stimuli, there are portable closure elements serving to cover, i.e. occlude, one eye of the viewer in each case. For the respectively non-occluded eye a portable eye tracking device 3 is available, with which characteristic values of the non-occluded eye can be measured.

The interaction of the eye tracking devices 3 and the portable closure elements 2 is controlled by a first control unit 5. For this purpose, this first control unit 5 has a first communication interface 140 to the eye tracking devices 3 and a second communication interface 150 to the closure elements 2. A display on the first display element 1 is controlled by a second control unit 6 that is connected to the first display element 1. The second control unit 6 has a third communication interface 160 to the first control unit 5 and controls the display on the display element 1 on the basis of the characteristic values detected by the portable eye tracking devices 3.

The closure elements 2 can be, for example electronically controllable liquid crystal shutters, also referred to as LC shutters. The eye tracking devices 3 can be, for example, portable video oculography devices, also referred to as VOG devices, infrared oculography devices, also referred to as IROG devices, or a combination of both.

To make the use of the apparatus 100 simple and pleasant to the viewer, the first control unit 5, the eye tracking device 3 and the closure elements 2 can be combined in one user unit, which can additionally be put on in the manner of a pair of glasses. To make the use of the apparatus 100 possible also for persons who have further eye complaints, the user unit can additionally have slots that are suitable for receiving refraction lenses 2a to be able to adapt the apparatus 100 to the optical power of the eye of the viewer.

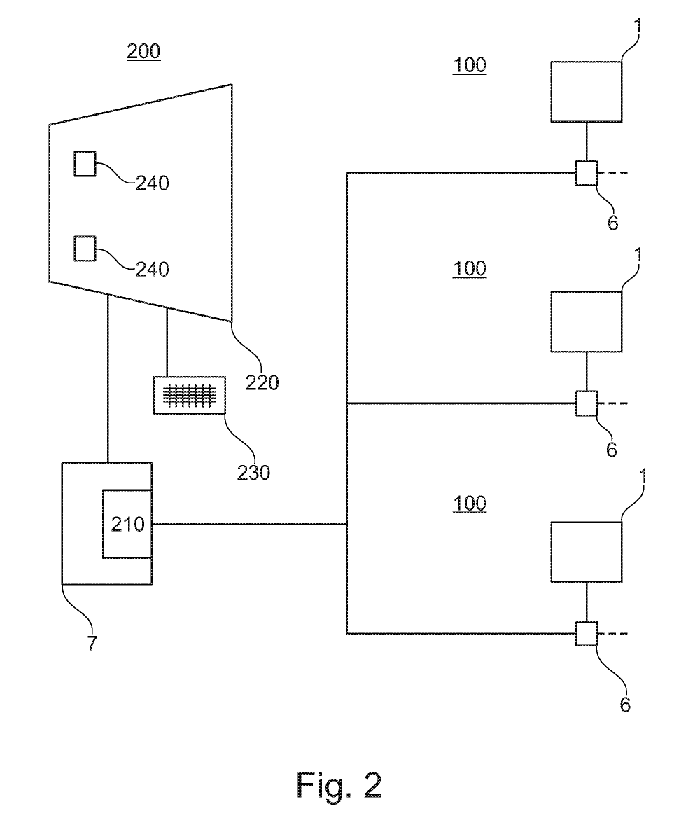

FIG. 2 shows a system showing the apparatus 100 represented in FIG. 1. Moreover, the system has a control device 200 which is connected to the respective second control units 6 of the apparatus 100 via a fourth communication interface 210, and which can communicate with the apparatus 100 via said connection. Further, for the operation of the control device 200 a second display element 220 and an input device 230 are connected. Via the communication connection operating data 240 of the apparatus 100 are transmitted to the control device 200. The operating data 240 were collected beforehand by the respective second control units 6 of the apparatus 100.

At the control device 200, the operating data 240 are represented on the second display device 220, and via the input device 230 inputs can be made that serve to control the apparatus 100. In the reverse direction of the previous data transmission of the operating data 240 control information 250 is sent by the control device 200 to the apparatus 100, received there by the second control units 6 and taken into account when operating the respectively particular apparatus 100.

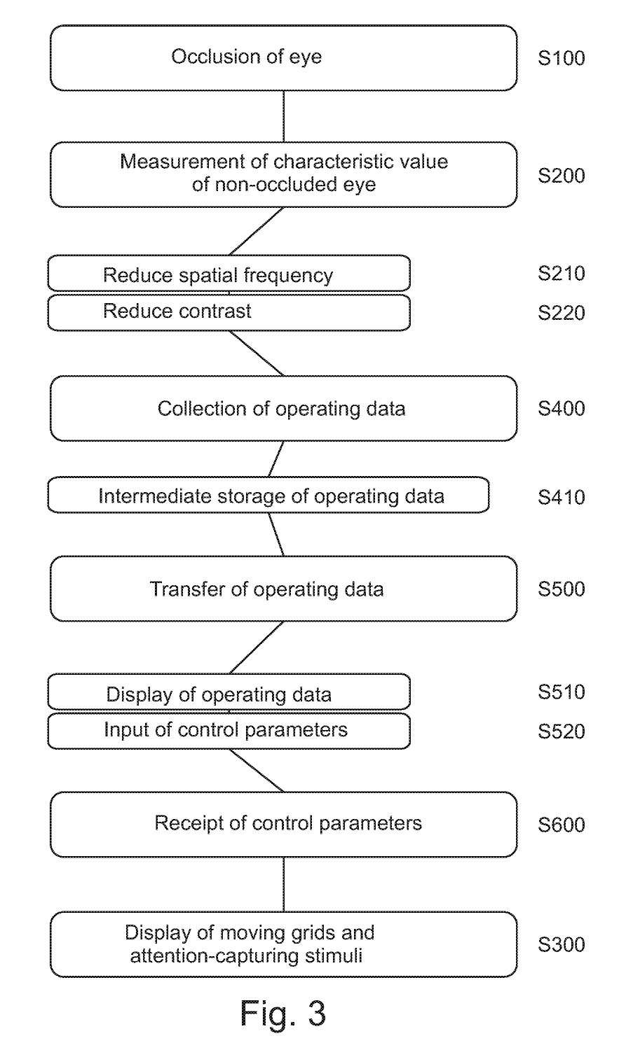

FIG. 3 shows a flow chart of a method for improving the visual function. Therein in step S100 an eye of the viewer is occluded, i.e. covered in lightproof fashion, by the portable closure element 2. Subsequently, in step S200 at least one characteristic value of the non-occluded eye of the viewer is measured by the eye tracking device 3. Finally, in step S300 the moving grids 110 and the attention-capturing stimuli 130 are displayed to a non-occluded eye on the first display element 1. Said display on the first display element 1 is controlled by the control unit 6 on the basis of the characteristic values measured by the eye tracking device 3. Among other things, the spatial frequency and the contrast of the display are controlled taking account of the measured characteristic values.

Upon measuring in step S200 for example two characteristic values can be measured by first reducing in step S210 the spatial frequency of the moving grids 110 on the first display element 1, until a monocular optokinetic nystagmus (MOKN) occurs. The value of the spatial frequency set at this time is stored as the first characteristic value.

Subsequently, in step S220 the contrast of the display of the moving grids 110 on the first display element 1 is reduced until the monocular optokinetic nystagmus (MOKN) disappears. The value of the contrast set at this time is stored as the second characteristic value.

A measurement according to step S200 is carried out for each eye. The measuring can be repeated for each eye with grids whose movement path is rotated by 90°, 180° and 270°. The course of the movement of the respective optokinetic nystagmus is recorded each time for the duration of its occurrence.

When the above-described system is utilized, in step S400 operating data 240 are collected by the control unit 6 of the apparatus 100 during the course of the procedure on the apparatus 100, and transferred to the control device 200 in step S500. The operating data 240 are possibly intermediately stored in step S410.

On the second display element 220 of the control device 200 in step S510, the operating data 240 transferred from the apparatus 100 are displayed, making it possible for a user of the control device 200 in step S520 to influence the course of the procedure in the apparatus 100 by making an input. The resulting input data, e.g. control parameters or control information 250, are sent in turn to the apparatus 100 in step S600, where the received control information 250 is then taken into account in step S300 when displaying the moving grids 110.

The apparatus 100 according to FIG. 1 can serve to train a fixation ability of persons (viewers), among them also amblyopics with impaired fixation ability. The apparatus 100 makes an improvement of the oculomotoric conduct possible, as well as (connected thereto) an increase of the visual acuity. Since in amblyopic persons, the monocular optokinetic nystagmi (OKN) are usually asymmetrical, the degree of asymmetry can be employed as an indirect measuring method for assessing the fixation ability, as well as the ability of binocular cooperation. The degree of asymmetry and its course during the use of the apparatus can be determined on the basis of the eye movement data with the aid of algorithms, and can be displayed via the second display element 220 of the control device 200. Test series have shown that a significant improvement of the visual function can occur after use for 3 months, if the apparatus is used daily for around 20 minutes.

The stimulation with the moving grids 110 that trigger the optokinetic nystagmus leads to an improvement of the fixation ability, thereby also increasing the visual acuity. This improvement also occurs when the occurrence of the optokinetic nystagmus is suppressed by fixation, for example by locating attention-capturing stimuli.

For (indirect) measuring, no input is expected from the viewer, but an involuntary movement pattern, namely the monocular optokinetic nystagmus of the eyes which is triggered by a corresponding optical stimulus in the form of moving grids, is measured with the aid of the eye tracking device 3 and the portable closure element 2.

In viewers with weak sight it can occur, in dependence on the type and scope of the lazy eye syndrome in comparison to emmetropic persons, that the triggering of the OKN occurs only in the case of moving grids 110 having a sufficiently low spatial frequency and sufficiently strong contrast. For amblyopic viewers (lazy eye syndrome in one eye or, more rarely, both eyes, due to an insufficient development of the visual system of the person during early childhood), this is applicable generally and depends on the severity of the amblyopia. Persons suffering from amblyopia are also referred to as amblyopics.

In contrast to emmetropic persons, pronounced asymmetries can occur between the MOKNs. Asymmetrical can mean, for example, that the slow phases differ in dependence on the movement direction of the moving grid 110. For the movement, there are respectively two directions: from the temple to the nose (from temporal to nasal) and reversely, from the nose to the temple (from nasal to temporal). A difference is therein that the angular speed of the eye differs in the slow phase.

These asymmetries also exist in emmetropic persons, but most frequently disappear as soon as a secure binocular cooperation was reached in the course of the post-natal ontogeny of the visual system. In amblyopics, however, this asymmetry remains in particular in the so-called amblyopic eye, with the result that the two MOKN of a person are different.

A grid stimulus that can trigger an optokinetic nystagmus can be complemented by a further stimulus in the foreground, which can attract the attention of the viewer. This results in a transient (temporary) fixation change, whereby, upon repeated presentation in the form of an exercise, the formation of a stable fixation change can be supported, which is frequently impaired in amblyopics.

Such a stimulus that can capture attention can be offered, for example, in the form of suitable games. The foreground stimulus must fulfill the condition that it does not permanently cover a large area of the grid stimulus (on average maximally up to 10%).

To sum up, it has been found that the OKN can on the one hand serve to assess the severity and type of an amblyopia with reference to an oculomotoric function, and on the other hand it can lead to a training effect that sustainably reduces the severity of the amblyopia in connection with attention-capturing foreground stimuli.

Reference numerals

| | |
|---|---|
| 1 | Display element |
| 2 | Portable closure element |
| 3 | Portable eye-tracking device |
| 5 | First control unit |
| 6 | Second control unit |
| 7 | Third control unit |
| 100 | Apparatus |
| 110 | Moving grids |
| 120 | Grid elements |
| 130 | Attention-capturing stimuli |
| 140 | First communication interface |
| 150 | Second communication interface |
| 160 | Third communication interface |
| 200 | Control device |
| 210 | Fourth communication interface |
| 220 | Second display element |
| 230 | Input device |
| 240 | Operating data |
| 250 | Control information |

What is claimed is:

1. An apparatus for improving a visual function, comprising
a first display element for displaying at least one moving grid having a plurality of grid elements;
at least one portable closure element for the occlusion of at least one eye;
a portable eye tracking device for measuring at least a monocular optokinetic nystagmus of a non-occluded eye;
a first control unit with a first communications interface to the eye tracking apparatus and a second communications interface to the at least one portable closure element; and
a second control unit connected to the first display element and comprising a third communication interface to the first control unit;
wherein the second control unit is connected to the first control unit and the second control unit is adapted to provide a display to the non-occluded eye of the at least one moving grid on the first display element and adjust spatial frequency of the at least one moving grid until a monocular optokinetic nystagmus occurs, and then reduce contrast of the at least one moving grid until the monocular optokinetic nystagmus disappears, and
wherein the second control unit is adapted to control display on the first display element of additional attention-capturing stimuli to the non-occluded eye.

2. The apparatus according to claim 1, wherein the at least one portable closure element is an electronically controllable liquid crystal shutter (LC shutter).

3. The apparatus according to claim 1, wherein the eye-tracking device is one of a portable video occulography device (VOG device), a portable infrared occulography device (IROG device), or a combination of the video occulography device and the infrared occulography device.

4. The apparatus according to claim 1, wherein the first control unit, the eye-tracking device and the at least one closure element are combined in a single user unit.

5. The apparatus according to claim 4, wherein the user unit can be put on like a pair of glasses.

6. The apparatus according to claim 4, wherein the user unit comprises slots for receiving refraction lenses.

7. A system for improving visual performance, comprising:
a first display element for displaying at least one moving grid having a plurality of grid elements;
at least one portable closure element for the occlusion of at least one eye;
a portable eye tracking device for measuring at least a monocular optokinetic nystagmus of a non-occluded eye;
a first control unit with a first communications interface to the eye tracking apparatus and a second communications interface to the at least one portable closure element;
a second control unit connected to the first display element and comprising a third communication interface to the first control unit, wherein the second control unit is adapted to provide a display to the non-occluded eye of the at least one moving grid on the first display element and adjust spatial frequency of the at least one moving grid until a monocular optokinetic nystagmus occurs, and then reduce contrast of the at least one moving grid until the monocular optokinetic nystagmus disappears, and wherein the second control unit is adapted to control display of additional attention-capturing stimuli to the non-occluded eye on the first display element; and
a control device having a third control unit, a second display element, at least one input device and at least a fourth communication interface to the second control unit;
wherein the second control unit[s] is further adapted to collect operating data for transfer to the third control unit, receive control information from the third control unit and to operate the first display unit, taking into account the control data received from the third control unit of the control device, and
wherein the third control unit is adapted to display on the second display element the transmitted operating data of the first display and is adapted to send control information based on an input from the at least one input device to the second control unit.

8. The system of claim 7, wherein the collected operating data from one of the second control unit[s] is temporarily stored in the second control unit.

9. A method for improving a visual function, comprising:
occlusion of an eye by means of a closure element;
measuring at least a monocular optokinetic nystagmus of a non-occluded eye by an eye-tracking device; and
displaying for a non-occluded eye moving grids and attention-capturing stimuli on a first display element and adjusting spatial frequency of the moving grids until a monocular optokinetic nystagmus occurs, and then reducing contrast of the at least one moving grid until the monocular optokinetic nystagmus disappears.

10. The method according to claim 9, further comprising:
collecting the operating data during the course of the method;
transferring the operating data to a control device;
displaying the transferred operating data on a display element of the control device;
entering an input to the control device; and
receiving from the control device the control information based on the input,
wherein the displaying is performed based on the control data received from the control means.

11. The method of claim 10, wherein the collecting of the operating data comprises intermediate storage of the operating data.

12. A computer program product stored on a non-transitory storage medium for improving a visual function, comprising:
a first logic for causing occlusion of an eye by means of a closure element
a second logic for measuring at least a monocular optokinetic nystagmus of a non-occluded eye by an eye-tracking device; and
a third logic for causing display to a non-occluded eye of moving grids and attention-capturing stimuli on a first display element, and adjusting spatial frequency of the moving grids until a monocular optokinetic nystagmus occurs, and then reducing contrast of the moving grids until the monocular optokinetic nystagmus disappears.
